(19) United States
(12) Patent Application Publication
(10) Pub. No.: US 2016/0303268 A1
(43) Pub. Date: Oct. 20, 2016

(54) Natural candle ointment in pourable container and related methods

(71) Applicant: Jessica Anderson Crocker, Glendale, CA (US)

(72) Inventor: Jessica Anderson Crocker, Glendale, CA (US)

(21) Appl. No.: 15/130,817

(22) Filed: Apr. 15, 2016

Related U.S. Application Data
(60) Provisional application No. 62/149,346, filed on Apr. 17, 2015.

(51) Int. Cl. A61L 9/03; A61K 47/44; A61K 41/00; A61K 36/28

(52) U.S. Cl. CPC A61L 9/037; A61K 36/28; A61K 47/44; A61K 41/0004

(57) ABSTRACT

Some embodiments of the present disclosure include a candle for providing a pourable ointment to a user. The candle may include an oil infused with at least one member selected from the group consisting of an herbal remedy and a homeopathic remedy. The infusion of the oil with the member may suitably create an ointment. The candle can also include a wax mixed with the oil, and optionally, a wick placed into the wax and oil mixture. When the candle is heated it may be configured to transform into a pourable ointment.

### CROSS-REFERENCE TO RELATED APPLICATIONS

This application claims the priority and benefit of U.S. Prov. Pat. App. Ser. No. 62/149,346 (filed Apr. 17, 2015) by Jessica Anderson Crocker and entitled “NATURAL CANDLE OINTMENT IN POURABLE CONTAINER.” The cited provisional application is hereby incorporated by reference in its entirety.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO AN APPENDIX SUBMITTED ON A COMPACT DISC AND AN INCORPORATED BY REFERENCE OF THE MATERIAL ON THE COMPACT DISC

Not applicable.

### STATEMENT REGARDING PRIOR DISCLOSURES BY THE INVENTOR OR A JOINT INVENTOR

Reserved for a later date, if necessary.

### BACKGROUND OF THE INVENTION

1. Field of Invention

The disclosed subject matter is in the field of candles. Further, embodiments described herein relate generally to ointments, and more particularly, to a natural candle comprising an ointment, wherein the natural candle may be stored in a pourable container and may have a wick that can be lit.

2. Background of the Invention

Ointments are needed for various reasons and, sometimes, people prefer applying ointments when they are warmed. Specifically, conventional ointments are often hard and cold and sometimes difficult to rub into the skin. Currently, massage candles exist, but there are no candles that comprise an ointment. Thus, existing candles do not soothe ailments and do not absorb into the skin well.

Therefore, what is needed is a candle that comprises an ointment, wherein when the candle is heated, it melts and can be poured onto a user's skin to apply the ointment to the skin.

### SUMMARY OF THE INVENTION

Some embodiments of the present disclosure include a candle for providing a pourable ointment to a user. The candle may include an oil that is infused with at least one member selected from the group consisting of an herbal remedy and a homeopathic remedy. The infusion of the remedy to the oil creates an ointment. The candle may also include a wax mixed with the oil or ointment. In a preferred embodiment, the mixture of wax and oil may be solidified. Optionally, the candle can feature a wick placed into the wax and oil mixture. When the candle is heated it may be configured to transform from the solidified state into a pourable ointment.

### BRIEF DESCRIPTION OF THE SEVERAL VIEWS OF THE DRAWINGS

Other objectives of the disclosure will become apparent to those skilled in the art once the invention has been shown and described. The manner in which these objectives and other desirable characteristics can be obtained is explained in the following description and attached figures in which:

FIG. 1 is a perspective view of a candle for providing a pourable ointment to a user; and,

FIG. 2 is a contextual view of the candle for providing a pourable ointment to a user of FIG. 1.

It is to be noted, however, that the appended figures illustrate only typical embodiments of this invention and are therefore not to be considered limiting of its scope, for the invention may admit to other equally effective embodiments that will be appreciated by those reasonably skilled in the relevant arts. Also, figures are not necessarily made to scale but are representative.

### DETAILED DESCRIPTION OF PREFERRED EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

Generally, the candle of the present disclosure may be used to provide light to an area or as a meltable and pourable ointment and may be comprised of the following elements.

### 1. Oil Based Ointment; 2. Wax.

This list of possible constituent elements is intended to be exemplary only, and it is not intended that this list be used to limit the candle of the present application to just these elements. Persons having ordinary skill in the art relevant to the present disclosure may understand there to be equivalent elements that may be substituted within the present disclosure without changing the essential function or operation of the device.

The various elements of the candle of the present invention may be related in the following exemplary fashion. It is not intended to limit the scope or nature of the relationships between the various elements and the following examples are presented as illustrative examples only.

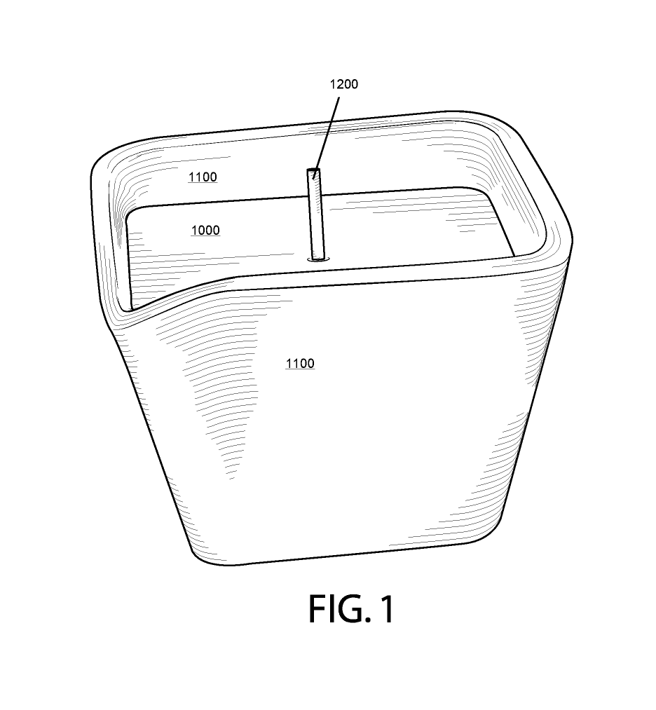

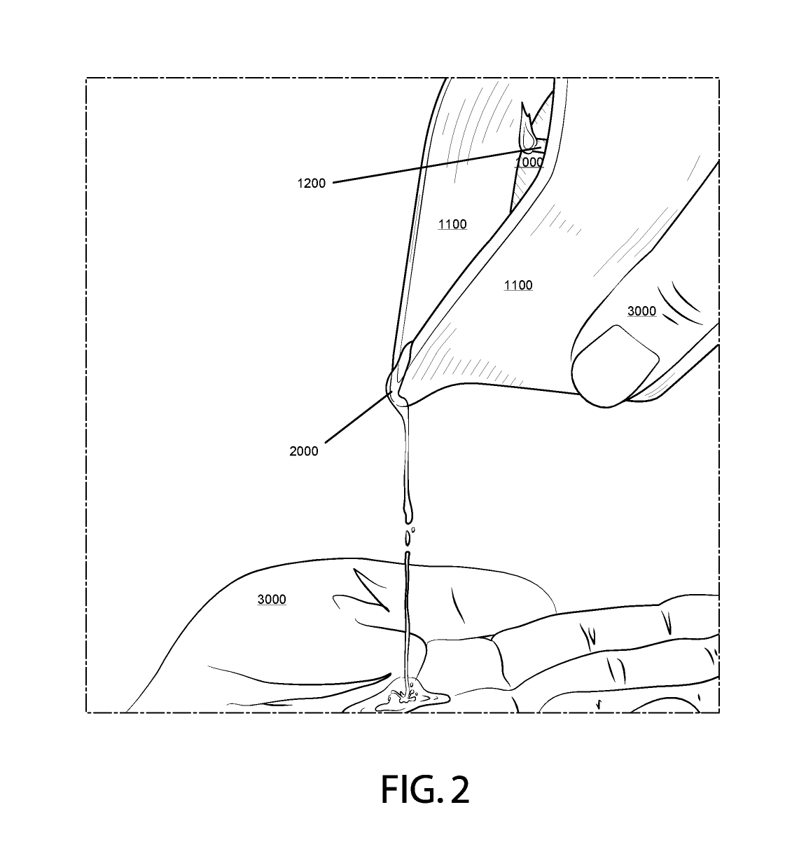

FIGS. 1 and 2 are respectively a perspective view and a contextual view of a candle 1000 for providing a pourable ointment 2000 (FIG. 2) to a user 3000 (FIG. 2). By way of example, some embodiments of the invention of the present disclosure comprise a candle 1000 for providing and applying an ointment 2000 (FIG. 2) to a user 3000 (FIG. 2). Referring to FIGS. 1 and 2, the candle 1000 may be provided within a pourable vessel 1100 and comprise a wax infused with an oil-based ointment. In some embodiments, the candle 1000 may further comprise a wick 1200 inserted into the candle 1000, wherein the wick 1200 is configured to be lit to melt the candle 1000 (see FIG. 2). Alternatively, the candle may be a wickless candle (not shown), wherein the candle may be warmed and melted using other heating methods, such as, a candle warmer (not shown).

The oil based ointment may comprise oil, such as coconut oil, infused with an herbal or homeopathic remedy. The oils may have a higher heat resistance than the oil. Suitable herbs may include, for example, chamomile and arnica. Suitable aromatherapy essential oil combinations may include, for example, eucalyptus. The wax may comprise a low melting point wax, such as beeswax or soy wax. However, the use of other herbs, homeopathic remedies, and oils are envisioned.

In a particular embodiment, coconut oil may be infused with chamomile and arnica to make the base of the candle a salve or ointment. About 20% beeswax or soy wax may then be added to make the candle solidify at room temperature. Then, an aromatherapy essential oil combination that is used traditionally to sooth or treat certain specific ailments may be added. Finally, the mixture may be poured into a candle container (see, e.g., FIG. 1), such as a container with a spout or an otherwise designed container that makes pouring easy, and a wick may be placed in the middle of the wax.

Referring again to FIGS. 1 and 2, to use the candle 1000, a user may light the wick 1200 or otherwise heat the candle 1000 to transform at least a portion of the candle 1000 into a liquid form 2000, which may then be poured onto and rubbed into the skin of a user 3000. The poured ointment 2000 may feel warm and soothing, and it may absorb easier than conventional ointments.

Although the method and apparatus is described above in terms of various exemplary embodiments and implementations, it should be understood that the various features, aspects and functionality described in one or more of the individual embodiments are not limited in their applicability to the particular embodiment with which they are described, but instead might be applied, alone or in various combinations, to one or more of the other embodiments of the disclosed method and apparatus, whether or not such embodiments are described and whether or not such features are presented as being a part of a described embodiment. Thus the breadth and scope of the claimed invention should not be limited by any of the above-described embodiments.

Terms and phrases used in this document, and variations thereof, unless otherwise expressly stated, should be construed as open-ended as opposed to limiting. As examples of the foregoing: the term “including” should be read as meaning “including, without limitation” or the like, the term “example” is used to provide exemplary instances of the item in discussion, not an exhaustive or limiting list thereof, the terms “a” or “an” should be read as meaning “at least one,” “one or more,” or the like, and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that might be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases might be absent. The use of the term “assembly” does not imply that the components or functionality described or claimed as part of the module are all configured in a common package. Indeed, any or all of the various components of a module, whether control logic or other components, might be combined in a single package or separately maintained and might further be distributed across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives might be implemented without confinement to the illustrated examples. For example, block diagrams and their accompanying description should not be construed as mandating a particular architecture or configuration.

All original claims submitted with this specification are incorporated by reference in their entirety as if fully set forth herein.

I claim:

1. A candle for providing a pourable ointment to a user, the candle comprising:
an oil infused with at least one remedy selected from the group consisting of an herbal remedy and a homeopathic remedy, creating an ointment;
a wax mixed with the oil; and
wherein when the candle is heated, said candle is configured to transform into the pourable ointment.

2. The candle of claim 1 where the oil is coconut oil.

3. The candle of claim 2 where the remedy is chamomile.

4. The candle of claim 2 where the remedy is arnica.

5. The candle of claim 2 where the remedy is chamomile and arnica.

6. The candle of claim 5 where the wax is beeswax.

7. The candle of claim 6 where the wax and infused oil when mixed is about 20% beeswax.

8. The candle of claim 5 where the wax is soy wax.

9. The candle of claim 6 where the wax and infused oil when mixed is about 20% soy wax.

10. The candle of claim 6 where the wax and oil, when mixed solidify at room temperature.

11. A method treating a user with a pourable ointment, said method comprising the steps of:
a. creating an ointment by infusing an oil with at least one remedy selected from the group consisting essentially of a herbal remedy and a homeopathic remedy;
b. mixing wax with said oil infused with said at least one remedy;
c. solidifying the wax and oil mixture in a pourable vessel with a wick extending from a surface thereof the wax and oil mixture;
d. starting the wick on fire;
e. waiting for a portion of the solidified wax and oil mixture to melt by the heat of the fire into a liquid, wherein said liquid is the pourable ointment; and,
f. pouring the pourable ointment by the pourable vessel; and
g. applying the pourable ointment to a user.

12. The method of claim 11 where the oil is coconut oil.

13. The method of claim 12 where the remedy is chamomile and arnica.

14. The method of claim 13 where the wax is beeswax.

15. The method of claim 14 where the wax and infused oil when mixed is about 20% beeswax.

16. The method of claim 13 where the wax is soy wax.

17. The method of claim 16 where the wax and infused oil when mixed is about 20% soy wax.

18. The method of claim 13 where the wax and oil, when mixed solidify at room temperature.

19. The method of claim 12 where the remedy is chamomile or arnica.

20. A method treating a user with a pourable ointment, said method comprising the steps of:
a. creating an ointment by infusing coconut oil with chamomile and arnica;
b. mixing bees wax or soy wax with said coconut oil infused with said chamomile and arnica;
c. placing a wick at middle position therein the wax and coconut oil mixture;
d. solidifying the wax and coconut oil mixture at room temperature in a pourable vessel with a spout;
e. starting the wick on fire;
f. waiting for a portion of the solidified wax and coconut oil mixture to melt by the heat of the fire into a liquid, wherein said liquid is the pourable ointment; and,
g. pouring the pourable ointment by the spout of the pourable vessel; and
h. applying the pourable ointment to a user.
